(19) United States
(12) Plant Patent Application Publication
(10) Pub. No.: US 2016/0324049 P1
(43) Pub. Date: Nov. 3, 2016

(54) Strawberry plant named 'Yoli'

(50) ;

(71) Applicant: John Larse, Aptos, CA (US)

(72) Inventor: John Larse, Aptos, CA (US)

(73) Assignee: Sweet Darling Sales, Inc., Aptos, CA (US)

(21) Appl. No.: 14/545,384

(22) Filed: Apr. 29, 2015

(51) Int. Cl. A01H 5/00

(52) U.S. Cl. PLT/208

(57) ABSTRACT

The present invention provides a new and distinct strawberry variety designated as ‘Yoli’ (a.k.a. ‘104522’).

### LATIN NAME OF THE GENUS AND SPECIES

Fragaria×ananassa

### VARIETAL DENOMINATIONS

‘Yoli’ (a.k.a. ‘104522’)

### BACKGROUND OF THE INVENTION

The present invention relates to a new and distinct strawberry variety designated as ‘Yoli’ (a.k.a. ‘104522’).

‘Yoli’ (a.k.a. ‘104522’) is the result of a controlled-cross between a female parent cultivar designated 1621 and a male parent cultivar designated 1285 (both unpatented, proprietary cultivars) made by the Inventor and was first fruited in Watsonville, Calif. growing fields. Following selection and during testing, the plant was originally designated ‘104522’ and subsequently named ‘Yoli’.

This new variety was asexually reproduced via runners (stolons) by the inventor at Watsonville, Calif. Asexual propagules from the original source have been tested in Watsonville growing fields and to a limited extent, grower fields in high elevation. The properties of this variety were found to be transmissible by such asexual reproduction. This cultivar is stable and reproduces true to type in successive generations of asexual reproduction.

### BRIEF SUMMARY OF THE INVENTION

This invention relates to new and distinctive strawberry cultivar designated as ‘Yoli’. These cultivars are primarily adapted to the climate and growing conditions of the central coast of California. This region provides the necessary temperatures required for it to produce a strong vigorous plant and to remain in fruit production from March through October. The nearby Pacific Ocean provides the needed humidity and moderate day temperatures and evening chilling to maintain fruit quality for the production months.

The following traits and photographs in combination distinguish strawberry variety ‘Yoli’ from known strawberry varieties. In addition, this new cultivar was confirmed to be a unique strawberry germplasm when tested against the California Seed & Plant Lab, Inc. (Elverta, Calif.) database using Short Sequence Repeats (SSRs). Plants for the botanical measurements in the present application were grown as annuals. Any color references are made to The Royal Horticultural Society Colour Chart, 1995 Edition, except where general terms of ordinary dictionary significance are used. The fruit produced by this new cultivar is attractive and of excellent quality.

### DESCRIPTION OF THE DRAWINGS

The accompanying color photographs depict various characteristics of the cultivars as nearly true as possible to make color reproductions.

FIG. 1 shows ‘Yoli’ plants growing in the field.

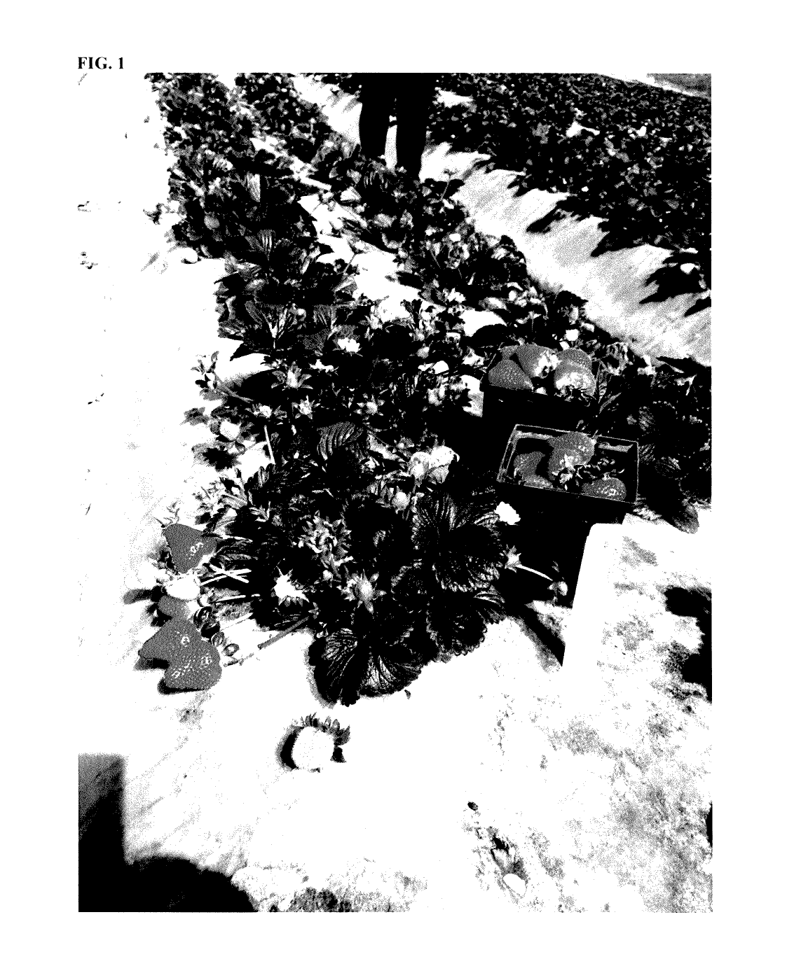

FIG. 2 shows inflorescences and fruits of ‘Yoli’ plants.

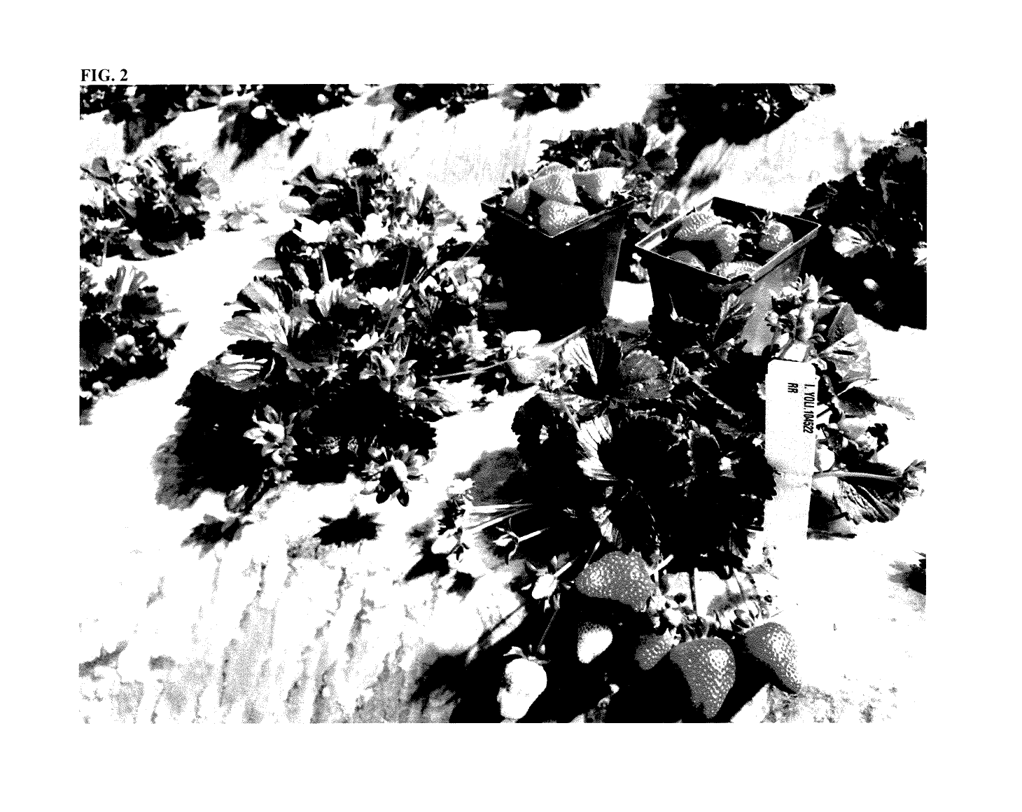

### DETAILED DESCRIPTION OF THE INVENTION

### ‘Yoli’ (a.k.a. ‘104522’)

This invention relates to a new and distinctive day-neutral type cultivar designated as ‘Yoli’. It is primarily adapted to the climate and growing conditions of the central coast of California. This region provides the necessary temperatures required for it to produce a strong vigorous plant and to remain in fruit production from March through October. The nearby Pacific Ocean provides the needed humidity and moderate day temperatures and evening chilling to maintain fruit quality for the production months.

The following traits in combination distinguish strawberry variety ‘Yoli’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

‘Yoli’ has not been observed under all possible environmental conditions, and the phenotype may vary significantly with variations in environment.

Hybrid Patent Data Sheet
(planted: October Year 1; measurement: June Year 2)
‘Yoli’ (a.k.a. ‘104522’)

| Plant | |
|---|---|
| Height (mm) | 310 |
| Diameter (mm) | 485 (the entire plant structure, including the |
| | “superstructure” of petioles, leaves, flowers) |
| | 60 (inner “base” or “heart” structure of the plant, |
| | i.e. the mature apical and axial crowns of the |
| | strawberry plant where petioles, flowers and |
| | runners are attached) |
| Terminal Leaf | |
| Length (mm) | 98 |
| Width (mm) | 99 |
| Blistering | |
| Petiole | |
| Length (mm) | 85 |
| Width (mm) | 3 |
| Petiolule | |
| Length (mm) | 242 |
| Width (mm) | 4 |
| Stolon | |
| Count (mm) | 43 |
| Diameter (mm) | 3 |
| Inflorescence | |
| Petal/flower | |
| Corolla (mm) | 15 |
| Calyx (mm) | 5 |
| Petal length (mm) | 17 |
| Petal width (mm) | 7 |
| Sepal length (mm) | 21 |
| Sepal width (mm) | 9 |
| Peduncle | |
| Stipules | |
| Fruit | |
| Fruit/truss | |
| Length (mm) | 36 |
| Width (mm) | 29 |
| Core length (mm) | 30 |
| Core width (mm) | 14 |

Achenes/fruit (measured only once)

| | |
|---|---|
| Glossiness | N/A |

1. A new and distinct cultivar of strawberry plant named ‘Yoli’ substantially as shown and described herein.
